(19) United States
(12) Patent Application Publication
(10) Pub. No.: US 2005/0233338 A1
(43) Pub. Date: Oct. 20, 2005

(54) Methods and compositions for assessing chromosome copy number

(76) Inventors: Michael T. Barrett, Mountain View, CA (US); Alicia F. Scheffer, Redwood City, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/828,892

(22) Filed: Apr. 20, 2004

(51) Int. Cl. C12Q 1/68; C12M 1/34

(52) U.S. Cl. 435/6; 435/287.2

(57) ABSTRACT

Methods and compositions for assessing chromosome copy number are provided. Specifically, the invention provides an array containing a plurality of features including at least one feature that contains oligonucleotides that are complementary to a structural region of a chromosome. The subject methods and compositions may be used to detect chromosome copy number in a cell, and, as such, may be employed in a variety of diagnostic and research applications. Kits and computer programming for use in practicing the subject methods are also provided.

### BACKGROUND OF THE INVENTION

Variations in the copy number of genomic sequences are associated with a variety of diseases and conditions. For example, gains and losses of genomic sequences up to and including whole chromosomes occur in many malignancies, e.g., colon cancer (Rajagopalan et al., Nature Cancer Review (2003) 3:695-701; Rabinovitch et al, Cancer Res. (1999) 59:5148-5153). As a result, tumor cells frequently have aneuploid genomes containing variable numbers of chromosomes and genetic content that deviates significantly from the normal diploid DNA content of non-neoplastic cells. Furthermore, genetic disorders frequently result from loss or gain of chromosomal regions. For example, in humans, trisomy of chromosome 21 results in Down's syndrome, trisomy of chromosome 13 results in Patau syndrome and abnormal numbers of sex chromosomes result in various developmental disorders, while abnormalities on the long arm of chromosome 18 are associated with 18q deletion syndrome.

Comparative genomic hybridization (CGH) is one approach that has been employed to evaluate variations in genomic copy number in cells. In one implementation of CGH, genomic DNA is isolated from normal reference cells, as well as from test cells (e.g., tumor cells). The two nucleic acids are differentially labeled and then simultaneously hybridized in situ to metaphase chromosomes of a reference cell. Chromosomal regions in the test cells which are at increased or decreased copy number can be identified by detecting regions where the ratio of signal from the two distinguishably labeled nucleic acids is altered. For example, those regions that have been decreased in copy number in the test cells will show relatively lower signal from the test nucleic acid than the reference compared to other regions of the genome. Regions that have been increased in copy number in the test cells will show relatively higher signal from the test nucleic acid.

In a recent variation of the above traditional CGH approach, the immobilized chromosome element has been replaced with a collection of solid support surface-bound polynucleotides, e.g., an array of surface-bound polynucleotides such as BAC (bacterial artificial chromosome) clones or cDNAs. Such approaches offer benefits over immobilized chromosome approaches, including a higher resolution, as defined by the ability of the assay to localize chromosomal alterations to specific areas of the genome.

However the variable numbers of individual chromosomes and the deviation from diploid DNA content in an aneuploid genome often makes it difficult to accurately identify and quantify regions of copy number changes in CGH analyses. Therefore, determining the copy number of each chromosome in a sample would increase the precision of these assays. Furthermore, a simultaneous measurement of ploidy and detection of individual regions of gains and losses would provide a more detailed analysis of a sample of interest. The copy number of a chromosome can be accurately evaluated by assessing the number of centromeres and/or telomeres of that chromosome in a cell. However, centromeres and telomeres of each chromosome in a genome typically contain or are associated with repetitive nucleic acid sequences, making them difficult to measure using current array-based CGH methods. The ability of array-based CGH methods to determine the copy number of entire chromosomes and the ploidy of a sample of interest remains limited.

As such, a great need still exists for array-based CGH methods that accurately evaluate chromosome copy numbers in a cell. This invention meets this, and other, needs.

Relevant Literature

United States Patents of interest include: U.S. Pat. Nos. 6,465,182; 6,335,167; 6,251,601; 6,210,878; 6,197,501; 6,159,685; 5,965,362; 5,830,645; 5,665,549; 5,447,841 and 5,348,855. Also of interest are published United States Application Serial No. 2002/0006622 and published PCT application WO 99/23256. Articles of interest include: Pollack et al., Proc. Natl. Acad. Sci. (2002) 99: 12963-12968; Wilhelm et al., Cancer Res. (2002) 62: 957-960; Pinkel et al., Nat. Genet. (1998) 20: 207-211; Cai et al., Nat. Biotech. (2002) 20: 393-396; Snijders et al., Nat. Genet. (2001) 29:263-264; Hodgson et al., Nat. Genet. (2001) 29:459-464; and Trask, Nat. Rev. Genet. (2002) 3: 769-778; Rabinovitch et al., Cancer Res. (1999) 59:5148-5153; Lee et al., Human Genet. (1997) 100:291:304.

### SUMMARY OF THE INVENTION

Methods and compositions for assessing chromosome copy number are provided. Specifically, the invention provides an array containing a plurality of features including at least one feature that contains oligonucleotides that are complementary to a chromosomal structural region, i.e., a centromeric or telomeric region of a chromosome. Chromosome copy number may be determined by labeling a chromosomal composition to make a population of labeled nucleic acids, and assessing binding of the labeled nucleic acids to the at least one chromosomal structural region feature of the array. The subject methods and compositions may be used to detect abnormal chromosome copy number in a cell, and, as such, may be employed in a variety of diagnostic and research applications. Kits and computer programming for use in practicing the subject methods are also provided.

### BRIEF DESCRIPTION OF THE FIGURES

The patent or application file contains at least one drawing executed in color. Copies of this patent application publication with color drawing(s) will be provided by the U.S. Patent and Trademark Office upon request and payment of the necessary fee.

FIG. 1 is a schematic representation of an embodiment of the subject invention.

FIG. 2 is a schematic representation of another embodiment of the subject invention.

FIGS. 3A and 3B are graphs showing examplary CGH data for chromosome 17 in the colon carcinoma cell line HT-29. FIG. 3a: Plot of log₂ fluorescence ratios along chromosome 17. FIG. 3b: Fluorescence signal intensities for chromosomal structural oligonucleotides.

FIG. 4 shows the karyotype for HT-29 and includes an enlarged view of chromosome 17.

### DEFINITIONS

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in many embodiments, are under 50 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of oligonucleotide target elements employed herein are present on a surface of the same planar support, e.g., in the form of an array.

The phrase “labeled population of nucleic acids” refers to mixture of nucleic acids that are detectably labeled, e.g., fluorescently labeled, such that the presence of the nucleic acids can be detected by assessing the presence of the label. A labeled population of nucleic acids is “made from” a chromosome sample, the chromosome sample is usually employed as template for making the population of nucleic acids.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” will be referenced as a moiety in a mobile phase (typically fluid), to be detected by “surface-bound polynucleotides” which are bound to the substrate at the various regions. These phrases are synonymous with the arbitrary terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound polynucleotides, as is commonly known in the art and described below, is not a mixture of capture agents because the species of surface-bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides, polypeptides and intact chromosomes of interest are well-known in the art and include, for example, ion-exchange chromatography, affinity chromatography, sorting, and sedimentation according to density.

The terms “assessing” and “evaluating” are used interchangeably to refer to any form of measurement, and include determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

If a surface-bound polynucleotide “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

### DESCRIPTION OF THE SPECIFIC EMBODIMENTS

Methods and compositions for assessing chromosome copy number are provided. Specifically, the invention provides an array containing a plurality of features including at least one feature that contains oligonucleotides that are complementary to a chromosomal structural region, i.e., a centromeric or telomeric region of a chromosome. Chromosome copy number may be determined by labeling a chromosomal composition to make a population of labeled nucleic acids, and assessing binding of the labeled nucleic acids to the at least one chromosomal structural region feature of the array. The subject methods and compositions may be used to detect abnormal chromosome copy number in a cell, and, as such, may be employed in a variety of diagnostic and research applications. Kits and computer programming for use in practicing the subject methods are also provided.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

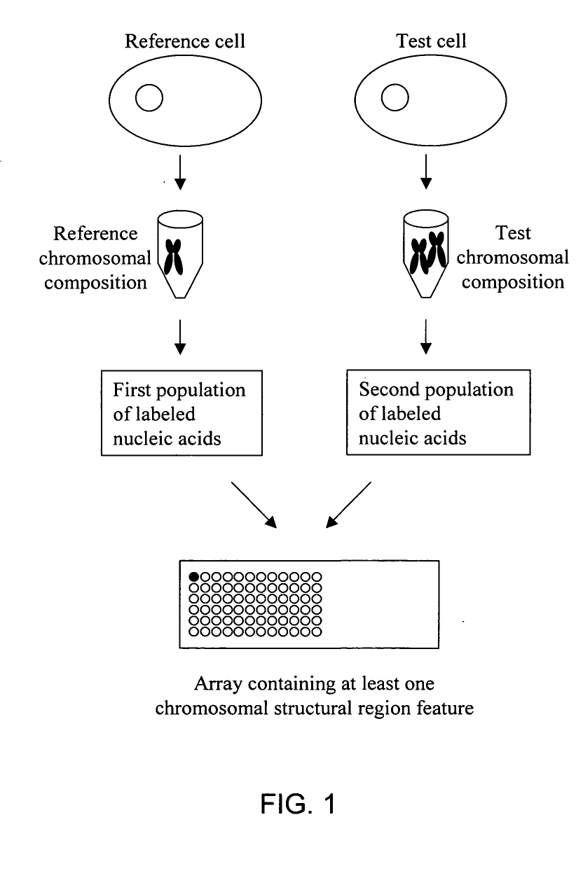

As summarized above, the present invention provides methods and compositions for assessing chromosome copy number in a cell. With reference to FIG. 1, showing an exemplary embodiment of the invention, the methods usually involve preparing both reference and test chromosomal compositions from a reference and test cell, respectively, making a first and second population of labeled nucleic acids using those compositions, and contacting the populations of nucleic acids with an array containing at least one chromosomal structural region feature.

In further describing the present invention, arrays containing at least one chromosomal structural region feature will be described first, followed by a detailed description of how the arrays may be used in the subject methods. Finally, representative kits and computer programming for use in practicing the subject methods will be discussed.

Arrays Containing at Least One Chromosomal Structural Region Oligonucleotide Feature

As mentioned above, the invention provides an array containing at least one chromosomal structural region oligonucleotide feature. By “chromosomal structural region oligonucleotide” is meant an oligonucleotide that corresponds to, i.e., has a sequence that is at least partially complementary to or the same as and will base-pair with, a structural region of a chromosome. Structural regions of a chromosome usually include centromeric and telomeric regions of a typical eukaryotic chromosome. In other words, chromosomal structural region oligonucleotides are oligonucleotides that specifically bind to structural regions of a chromosome, e.g., telomeric or centromeric regions of a chromosome, under stringent binding conditions. Accordingly, chromosomal structural region oligonucleotides usually have a sequence that is present in a centromere region or telomere region of a chromosome, and may be used to detect a particular chromosome. In most embodiments, and as will be discussed in greater detail below, chromosomal structural region oligonucleotides are generally chromosome-specific in that they correspond to and may be used to detect a single chromosome in a cell, even when other chromosomes are present.

In certain embodiments, the arrays of the invention may contain “chromosomal non-structural region oligonucleotides”. Such oligonucleotides generally correspond to regions of a chromosome that are not structural regions, e.g., not centromere or telomere regions. Such non-structural regions of a chromosome normally lack the repetitive elements found in telomere and centromere regions and are usually unique to a genome.

A “chromosomal structural region oligonucleotide feature”, therefore, is a feature of an array, i.e., a spatially addressable area of an array, as described above, that contains a plurality of surface-bound chromosomal structural region oligonucleotides. Accordingly, a feature contains “surface-bound” oligonucleotides that are bound, usually covalently, to an area of an array. In most embodiments a single type of oligonucleotide is present in each chromosomal structural region oligonucleotide feature (i.e., all the oligonucleotides in the feature have the same sequence). However, in certain embodiments, the oligonucleotides in a feature may be a mixture of oligonucleotides with different sequence.

In general, the subject features contain oligonucleotides that are about 10 to about 200 bases, however, in certain embodiments, the oligonucleotides may be about 10 to about 100 bases, about 10 to about 80 bases, about 10 to about 50 bases, or about 10 to about 30 bases in length. In particular embodiments, the subject features contain oligonucleotides are 20-60 bases in length.

The subject arrays may contain a single chromosomal structural region oligonucleotide feature. However, in many embodiments, the subject arrays may contain more than one such feature, and those features may correspond to (i.e., may be used to detect) a plurality of different chromosomes of a cell. Accordingly, the subject arrays may contain a plurality of features (i.e., 2 or more, about 5 or more, about 10 or more, about 15 or more, about 20 or more, about 30 or more, about 50 or more, about 100 or more, about 150 or more, about 200 or more, about 500 or more, usually up to about 1000 or about 10,000 or more features, etc.), each containing a different chromosomal structural region oligonucleotide or combinations of structural and nonstructural chromosome regions oligonucleotides. In certain embodiments, the subject arrays contain a plurality of subject oligonucleotide features that correspond to a plurality of cellular chromosomes. Accordingly, a subject array may contain chromosomal structural region oligonucleotide features that correspond to 1, more than about 5, more than about 10, more than about 15, more than about 20, more than about 25, more than about 30, more than about 35, more than about 40, more than about 50, or more than about 66, usually up to about the haploid number of chromosomes of interest in a cell. In particular embodiments, therefore, the subject arrays may contain chromosomal structural region oligonucleotide features for, i.e., corresponding to, all of the autosomal chromosomes of a cell, plus, optionally, any sex chromosomes. For example, in the case of human cells, the subject arrays may contain at least a chromosomal structural region oligonucleotide feature for each of the 22 human autosomes, plus a chromosomal structural region oligonucleotide feature for the X and Y chromosomes.

In particular embodiments, a subject array may contain features for a pair of structural regions of a single chromosome, e.g., a pair of structural regions such as a centromere region and a telomere region of a single chromosome. Accordingly, a subject array may contain a plurality of chromosomal structural region oligonucleotide feature pairs (e.g., 2, about 5 or more, about 10 or more, about 15 or more, about 20 or more, etc., as above), each pair of features corresponding to a different chromosome of interest. Further, since each chromosome of a cell usually contains two telomeres (one for each of the p and q arms) and one centromere, in particular embodiments, the subject arrays may contain at least one chromosomal centromeric region oligonucleotide feature and at least two chromosomal telomeric structural region oligonucleotide features for each chromosome of interest.

Finally, the subject arrays may contain a plurality of chromosomal structural region oligonucleotide features for a particular chromosome. For example, the subject arrays may contain more than 2, about 5 or more, about 10 or more, about 20 or more, usually up to about 50 or 100 or more, different chromosomal structural region oligonucleotide features (i.e. features containing oligonucleotides of different sequence) for each chromosome if interest.

In many embodiments, the subject oligonucleotides contained in the subject features have been designed according to one or more particular parameters to be suitable for use in a given application, where representative parameters include, but are not limited to: length, melting temperature (TM), non-homology with other regions of the genome, hybridization signal intensities, kinetic properties under hybridization conditions, etc., see e.g., U.S. Pat. No. 6,251,588, the disclosure of which is herein incorporated by reference. In certain embodiments, the entire length of the subject oligonucleotides is employed in hybridizing to centromeric or telomeric region in a genome of interest, while in other embodiments, only a portion of the subject oligonucleotide has sequence that hybridizes to sequence found in a genome of interest, e.g., where a portion of the oligonucleotide serves as a tether. For example, a given oligonucleotide may include a 30 nt long genome specific sequence linked to a 30 nt tether, such that the oligonucleotide is a 60-mer of which only a portion, e.g., 30 nt long, is genome specific.

Surface-bound telomeric and centromeric oligonucleotides, and array platforms upon which the subject chromosomal structural region oligonucleotide features exist, are described in greater detail below.

Centromeric Oligonucleotides

A centromeric oligonucleotide is an oligonucleotide that corresponds to, i.e., hybridizes to and may be used to detect, a centromere region of a particular chromosome. In most embodiments, such an oligonucleotide is specific for a centromere region of a particular chromosome, i.e., is “chromosome-specific”, in that it can detect the centromere of that chromosome, even in the presence of other chromosomes. In other words, a centromere oligonucleotide contains a nucleic acid sequence that is present in a centromeric region of a particular chromosome, where a centromeric region contains a centromere of a particular chromosome, plus sequences immediately flanking (i.e., within about 1, 2, 3, 4 or 5 cM of, or within about 0.1, 0.5, about 1, about 2 or about 3 mega basepairs of) that centromere.

In general, since the human genome and the genomes of several other model species (e.g., mouse, Drosophila, Arabidopsis, Saccharomyces, etc.) have been sequenced, the sequences of centromere regions of all chromosomes for many cells of interest are described in the art. In particular, centromeric sequences that are unique to each of the human chromosomes are generally well understood in the art (see, e.g., Lee et al., Human Genet. (1997) 100:291-304) and used in fluorescence in situ hybridization (FISH) studies of chromosomes, and, as such, need not be described here in any great detail.

In particular embodiments, the subject centromeric oligonucleotides correspond to unique repetitive sequences found in the centromeric regions of chromosomes. For example, the centromeric oligonucleotides may correspond to “alphoid” or “alpha-satellite” DNA, which is present at the centromeric region of every chromosome of an animal cell with a sequence that is different for each chromosome (see, e.g., Lee et al., Human Genet. (1997) 100:291-304 and Jabs et al., Am. J. Hum. Genet. (1987) 41:374-90). In many embodiments, the sequence of at least a portion, e.g., about 15-20 nt, about 20-50 nt, or about 25-80 nt) of subject oligonucleotides is encompassed by the repeated sequence.

Nucleic acids that hybridize to each of the centromeres of all of the human chromosomes may be purchased as “CEP Probes” from Vysis Inc. (Downers Grove, Ill.), or as “Human Chromosome-Specific Centromeric Probes”, from Open Biosystems (Huntsville, Ala.). Alternatively chromosome specific centromeric oligonucleotides may be designed using known sequences. For example, the probes discussed in the following publications may be used to design suitable centromeric oligonucleotides for each of the human chromosomes: chromosome 1: Waye et al., (Genomics (1987) 1:43-51); Hardas et al., (Genomics (1994) 21:359-63); Solus et al., (Somat. Cell. Mol. Genet. (1988) 14:381-91); chromosome 2: Ostroverkhova et al., (Am J Med Genet. (1999) 87:217-20; Matera et al., (Hum Mol Genet. (1992) 1:535-9); chromosome 3: Delattre et al., Hum Hered. (1988) 38:156-67; Varella-Garcia et al., (Cancer Res. (1998) 58:4701-7); chromosome 4: Grimbacher et al., (Genet. Med. (1999) 1:213-8); chromosome 5: Matera et al., (Genomics (1993) 18:729-31); Reichenbach et al., (Am. J. Med. Genet. (1999) 85:447-51) chromosome 6: Lastowska et al., Cancer Genet. Cytogenet. (1994) 77:99-105); chromosome 7: Mark et al., (Exp Mol Pathol. (1999) 67:109-17); Zhao et al. (Ann. Clin. Lab. Sci. (1998) 28:51-6); Jenkins et al., (Cancer Res. (1998) 58:759-66); chromosome 8: Zhao et al., (Ann. Clin. Lab. Sci. (1998) 28:51-6); Macoska et al., (Urology (2000) 55:776-82); Mark et al., (Exp. Mol. Pathol. (1999) 66:157-62); chromosome 9: Rocchi et al., (Genomics (1991) 9:517-23); Gutierrez-Angulo et al., (Genet Couns. (2001) 12:359-62); chromosome 10: Wang et al., (Somat. Cell Mol. Genet. (1996) 22:241-4); Devilee et al., (Genomics (1988) 3:1-7); Howe et al., (Hum Genet. (1993) 91:199-204); chromosome 11: Voorter et al., (Int. J. Cancer (1996) 65:301-7); Kraggerud et al., (Cancer Genet. Cytogenet. (2003) 147:1-8); chromosome 12: Looijenga et al., (Cytogenet. Cell Genet. (1990) 53:216-8); Zhao et al., (Ann. Clin. Lab. Sci. (1998) 28:51-6); chromosome 13: Warren et al., (Genomics (1990) 7:110-4); chromosome 14: Earle et al., (Cytogenet Cell Genet. (1992) 61:78-80); chromosome 15: Stergianou et al., (Hereditas (1993) 119:105-10); chromosome 16: Greig et al., (Am. J. Hum. Genet. (1989) 45:862-72); chromosome 17: Fink et al., (Hum Genet. (1992) 88:569-72); chromosome 18: Verma et al., (Ann Genet. (1998) 41:154-6); chromosome 19: Hulsebos et al., (Cytogenet. Cell Genet. (I 988) 47:144-8); chromosome 20: Meloni-Ehrig et al., Cancer Genet. Cytogenet. (1999) 109:81-5); chromosome 21: chromosome 21: Maratou et al., (Genomics (1999) 57:429-32); Verma et al., (Clin. Genet. (1997) 51:91-3); X chromosome: Yang et al., (Proc. Natl. Acad. Sci. (1982) 79:6593-7); Crolla et al., (Hum. Genet. (1989) 81:269-72); and Y chromosome: Davalos et al., (Am. J. Med. Genet. (2002) 111:202-4); Rivera et al., (Ann. Genet. (1996) 39:236-9); Tho et al., (Am. J. Obstet. Gynecol. (1988) 159:1553-7). Hybridization conditions for these probes may require higher stringency than used for the nonstructural chromosome probes on the array. Therefore, higher stringency structural probes may be contained on a separately hybridizable part of the array. Alternatively different chemistries may be used to synthesize or modify probes such that they hybridize to their appropriate targets under similar stringency conditions as the nonstructural probes on the array.

Telomeric Oligonucleotides

A telomeric oligonucleotide is an oligonucleotide that corresponds to, i.e., hybridizes to and may be used to detect, a telomere region of a particular chromosome. In most embodiments, such an oligonucleotide is specific for a telomere region of a particular chromosome, i.e., is “chromosome-specific”, in that it can detect the telomere of that chromosome, even in the presence of other chromosomes. In other words, a telomeric oligonucleotide contains a nucleic acid sequence that is present in a telomeric region of a particular chromosome, where a telomeric region contains a telomere of a particular chromosome, plus sequences immediately flanking that telomere.

In general, since the human genome and the genomes of several other model species (e.g., mouse, Drosophila, Arabidopsis, Saccharomyces, etc.) have been sequenced, the sequences of telomere regions of all chromosomes for many cells of interest are described in the art. In particular, chromosome-specific telomeric regions are generally well understood in the art and used in FISH studies of chromosomes, and, as such, need not be described here in any great detail.

Since all of the chromosomes of a cell usually have the same telemeric repeat sequence (which, in the case of humans is d(TTAGGG)n), the subject telomeric oligonucleotides usually correspond to sequences that are immediately adjacent to the telomere (i.e., sequences that are less then about 100 kb, less then about 200 kb, less then about 300 kb, less than about 500 kb or less than about 1 Mb from, or up to 1 cM from the end of a chromosome). Accordingly, the subject telomeric oligonucleotides may correspond to subtelomeric regions (Riethman, et al., Genome Research 14:18-28 (2004)), which term is well recognized in the art. These regions include both repetitive and unique sequences. Probes corresponding to the unique sequences in these regions could be designed and selected such that they may be hybridized under similar conditions as the non-structural probes. Thus telomeric oligonucleotides may or may not be included in the same hybridizable part of the array containing the non-structural probes.

Accordingly, chromosome-specific telomeric oligonucleotides may be designed using telomeric sequences that are well known in the art. For example, complete sets of telomeric probes for human chromosomes are described in NIH/IMM Collaboration, (Nature Genetics (1996) 14:86); Knight et al., (Am. J. Hum. Genet. (2000) 67:320-332); Knight et al., (J. Med. Genet. (2000) 37:401-4091) and Veltman et al., (Am. J. Hum. Genet. (2002) 70:1269-76). Further, complete sets of telomeric probes for human chromosomes may be purchased from Vysis Inc. (Downers Grove, Ill.; as ToTelVysion and TelVysion chromosome-specific telomere probes) and from Open Biosystems (Huntsville, Ala.; as Human Chromosome Telomeric Region Probes).

Arrays Platforms for Assessing Chromosome Copy Number

The subject chromosomal structural region oligonucleotide features are usually present in an array of oligonucleotide features. In general, arrays suitable for use in performing the subject methods contain a plurality (i.e., at least about 100, at least about 500, at least about 1000, at least about 2000, at least about 5000, at least about 10,000, at least about 20,000, usually up to about 100,000 or more) of addressable features containing oligonucleotides that are linked to a usually planar solid support. Features on a subject array usually contain polynucleotides that hybridize to, i.e., bind to, genomic sequences from a cell. Accordingly, such “comparative genome hybridization arrays”, for short “CGH arrays” typically have a plurality of different oligonucleotides that are addressably arrayed. In certain embodiments, the subject array features may also contain other polynucleotides, such as cDNAs, or inserts from phage BACs or plasmid clones. As such, while the subject CGH arrays usually contain features of oligonucleotides, they may also contain features of polynucleotides that are about 201-5000 bases in length, about 5001-50,000 bases in length, or about 50,001-200,000 bases in length, depending on the platform used.

If other polynucleotide features are present on a subject array, they may be interspersed with, or in a separately-hybridizable part of the array from, the subject oligonucleotides.

In particular embodiments, each centromere or telomere region of interest is represented by at least 10, 20 or 30 or more, usually up to about 50 features containing oligonucleotides of different, non-overlapping, or, in some embodiments, overlapping, sequence.

In general, methods for the preparation of polynucleotide arrays are well known in the art (see, e.g., Harrington et al,. Curr Opin Microbiol. (2000) 3:285-91, and Lipshutz et al., Nat Genet. (1999) 21:20-4) and need not be described in any great detail.

Arrays can be fabricated using any means, including drop deposition from pulse jets or from fluid-filled tips, etc, or using photolithographic means. Either polynucleotide precursor units (such as nucleotide monomers), in the case of in situ fabrication, or a previously synthesized polynucleotides (e.g., oligonucleotides, amplified cDNAs or isolated BAC, bacteriophage and plasmid clones, and the like) can be deposited. Such methods are described in detail in, for example U.S. Pat. Nos. 6,242,266, 6,232,072, 6,180,351, 6,171,797, 6,323,043, etc.

Methods for Assessing Chromosome Copy Number

The invention provides a method for assessing chromosome copy number in a cell. In general, the method involves labeling, e.g., distinguishably labeling, two genomic samples to produce a first and second population of labeled nucleic acids, and assessing binding of the labeled nucleic acids to a subject feature, i.e., a chromosomal structural region oligonucleotide feature. In many embodiments, the methods generally follow the methods that are well known in the art and described in, e.g., Pinkel et al., (Nat. Genet. (1998) 20:207-211); Hodgson et al., (Nat. Genet. (2001) 29:459-464); and Wilhelm et al., (Cancer Res. (2002) 62: 957-960), except that an accurate determination of chromosome copy number may be assessed by evaluating binding to the subject feature.

With specific reference to FIG. 1, in most embodiments, “chromosomal compositions” from a test cell and a reference cell are usually provided, where a “chromosomal composition” is a composition containing chromosomes of a cell, or a nucleic acid derivative thereof. For example, a chromosomal composition may contain the entire complement of chromosomes of a cell (i.e., the chromosomes that make up the genome of a cell), fragmented versions thereof, amplified copies thereof, or amplified fragments thereof. In particular embodiments, therefore, a chromosomal composition contains amplified regions of a cellular genome, e.g., centromere or telomere regions that hybridize to particular surface-bound centromere or telomere oligonucleotides. Accordingly, a chromosome composition may be of reduced complexity (usually at least at 20-fold less e.g., 25-fold less, at least about 50-fold less, at least about 75-fold less, at least about 90-fold less, or at least about 95-fold less complex in terms of total numbers of sequences present in the chromosome composition as compared to the entire chromosome complement of a cell, up to and including a single centromeric or telomeric region being represented in the composition) as compared to the entire complement of chromosomes of a cell. In most embodiments, the chromosomal regions of interest of a chromosomal sample (i.e., those regions that will hybridize to any subject oligonucleotides present on an array to be used), are generally concentrated in a reduced-complexity sample, relative to other regions of a genome.

The test and reference cells of a test and reference cell pair may be any two cells. However, in many embodiments, one cell of the pair has or is suspected of having a different phenotype compared to the other cell. In a particular embodiment, test and reference cell pairs include cancerous cells, e.g., cells that exhibit increased genomic instability, and non-cancerous cells, respectively or cells obtained from a sample of tissue from a test subject, e.g., a subject suspected of having a chromosome copy number abnormality, and cells obtained from a normal, reference subject, respectively.

In general, the cells used in the subject methods may be any cell of interest, including cells that contain or are suspected of containing an abnormal chromosome copy number. Accordingly, cells from yeast plants and animals, such as fish, birds, reptiles, amphibians and mammals may be used in the subject methods. In certain embodiments, mammalian cells, i.e., cells from mice, rabbits, primates, or humans, or cultured derivatives thereof, may be used.

In practicing the subject methods and with reference to FIG. 1, the chromosomal compositions are labeled to provide at least two different populations of labeled nucleic acids that are to be compared. The populations of nucleic acids may be labeled with the same label or different labels, depending on the actual assay protocol employed. For example, where each population is to be contacted with different but identical arrays, each nucleic acid population or collection may be labeled with the same label. Alternatively, where both populations are to be simultaneously contacted with a single array of surface-bound oligonucleotides, i.e., cohybridized, to the same array of immobilized nucleic acids, solution-phase collections or populations of nucleic acids that are to be compared are generally distinguishably or differentially labeled with respect to each other.

In general, the chromosome compositions may be distinguishably labeled using methods that are well known in the art (e.g., primer, extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The compositions are usually labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.) and POPRO3 TOPRO3 (Molecular Probes, Eugene, Oreg.). Further suitable distinguishable detectable labels may be found in Kricka et al. (Ann Clin Biochem. 39:114-29, 2002).

In certain embodiments, a population of labeled nucleic acids may be one that is of reduced complexity as compared to the initial chromosomal composition. By reduced complexity is meant that the complexity of the produced collection of probe nucleic acids is at least about 20-fold less, such as at least about 25-fold less, at least about 50-fold less, at least about 75-fold less, at least about 90-fold less, at least about 95-fold less complex, than the complexity of the initial chromosome composition, in terms of total numbers of sequences found in the produced population of labeled nucleic acids as compared to the initial chromosome composition, up to and including a single centromere or telomere region being represented in the population. The reduced complexity can be achieved in a number of different manners, such as by using sequence specific primers in the generation of labeled nucleic acids, and, as discussed above, by reducing the complexity of the chromosomal composition used to prepare the population of labeled nucleic acids, etc. Any convenient labeling protocol, such as the above described representative protocols, may be employed, where the protocols are adapted to provide for the desired reduced complexity, e.g., by using gene specific instead of random primers. Again, in most embodiments, chromosomal regions of interest of a chromosomal sample (i.e., those regions that will hybridize to any subject oligonucleotides present on an array to be used), are generally concentrated in a reduced-complexity sample, relative to other regions of a genome. In some embodiments, therefore, such regions of interest may be amplified relative to other regions in reducing the complexity of a sample.

In yet other embodiments, the population of labeled nucleic acids does not have reduced (i.e., has non-reduced) complexity.

Accordingly, using the above protocols, at least a first population of labeled nucleic acids and a second population of labeled nucleic acids are produced from two different chromosomal samples, e.g., a reference and test genomic samples, from two different cells. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different populations of labeled probe nucleic acids are labeled with the same label, such that they are not distinguishably labeled. In yet other embodiments, a feature of the different populations of labeled nucleic acids is that the first and second labels are typically distinguishable from each other. The constituent probe members of the above produced collections typically range in length from about 50 to about 10,000 nt, such as from about 200 to about 10,000 nt, including from about 50 to 1,000 nt, from about 50 to about 500, etc.

The labeling reactions produce a first and second population of labeled nucleic acids that correspond to the test and reference chromosome compositions, respectively. After nucleic acid purification and any pre-hybridization steps to suppress repetitive sequences (e.g., hybridization with Cot-1 DNA), the populations of labeled nucleic acids are usually contacted to an array of surface-bound polynucleotides, as discussed above, under conditions such that nucleic acid hybridization to the surface-bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

Accordingly, in the next step of the subject methods, and with reference to FIG. 1, the populations of labeled nucleic acids produced by the subject methods are contacted to a subject array under conditions such that nucleic acid hybridization to surface-bound oligonucleotides can occur. The collections can be contacted to the surface immobilized elements either simultaneously or serially. In many embodiments the nucleic acids are contacted with a subject array simultaneously. Depending on how the populations are labeled, the populations may be contacted with the same array or different arrays, where when the populations are contacted with different arrays, the different arrays are substantially, if not completely, identical to each other in terms of feature content and organization.

Standard hybridization techniques (using high stringency hybridization conditions) are used to probe subject array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos: 6,335,167; 6,197,501; 5,830,645; and 5,665,549; the disclosures of which are herein incorporate by reference.

Generally, nucleic acid hybridizations comprise the following major steps: (1) provision of an array of surface-bound polynucleotides; (2) pre-hybridization treatment to increase accessibility of surface-bound polynucleotides, and to reduce nonspecific binding; (3) hybridization of a population of labeled nucleic acids to the surface-bound polynucleotides, typically under high stringency conditions; (4) post-hybridization washes to remove nucleic acids not bound in the hybridization; and (5) detection of the hybridized nucleic acids. The reagents used in each of these steps and their conditions for use vary depending on the particular application.

Optionally, prior to step (3), the complexity of the population of labeled nucleic acids may be reduced by a pre-incubation, e.g., hybridized with nucleic acids to suppress repetitive or unwanted sequences. In some embodiments, Cot-1 nucleic acids may be used. However, in certain embodiments were it is desirable to suppress certain repetitive sequences but not others, the population of labeled nucleic acids may by pre-incubated with certain types of nucleic acids for suppressing only those undesirable sequences. For example, if labeled nucleic acids containing alpha-satellite sequences are deemed desirable (e.g., in the case where surface-bound oligonucleotides for centromeric alpha-satellite sequences are present in the array to be contacted, for example), the population of labeled nucleic acids may be incubated with a mixture of nucleic acids containing other repetitive sequences, e.g., Alu, LINEs (e.g., LINE-1), SINEs (e.g., SINE B1 and B2), and microsatellite repeat sequences (e.g. di-, tri-, tetra-, and pentanucelotide repeats) in the absence of alpha-satellite sequences.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between surface-bound oligonucleotides and complementary labeled nucleic acids in a sample. Representative high stringency assay conditions that may be employed in these embodiments are provided above.

The above hybridization step may include agitation of the immobilized targets and the sample of labeled nucleic acids, where the agitation may be accomplished using any convenient protocol, e.g., shaking, rotating, spinning, and the like.

In embodiments that use arrays with two separate hybridizable parts (i.e. one containing features specific for structural regions and the other containing features for other genome regions of interest) the hybridization conditions can be different for each part of the array. These conditions can include different hybridization buffers, different stringencies, and different hybridization times.

Following hybridization, the surface of immobilized nucleic acids is typically washed to remove unbound labeled nucleic acids. Washing may be performed using any convenient washing protocol, where the washing conditions are typically stringent, as described above.

Following hybridization and washing, as described above, the hybridization of the labeled nucleic acids to the array is then detected using standard techniques so that the surface of the array, is read. Reading of the resultant hybridized array may be accomplished by illuminating the array and reading the location and intensity of resulting fluorescence at each feature of the array to detect any binding complexes on the surface of the array. For example, a scanner may be used for this purpose that is similar to the AGILENT MICROARRAY SCANNER available from Agilent Technologies, Palo Alto, Calif. Other suitable devices and methods are described in U.S. patent applications: Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came)).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In certain embodiments, a chromosome copy number is assessed by determining a level of binding of the population of labeled nucleic acids to one or more chromosomal structural region oligonucleotide features corresponding to that chromosome. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of labeled nucleic acid to a chromosomal structural region oligonucleotide feature is proportional to the level of bound label, the level of binding of labeled nucleic acid is usually determined by assessing the amount of label associated with the feature.

In certain embodiments, a chromosome copy number may be assessed by evaluating binding of one or more chromosomal structural region oligonucleotide features corresponding to that chromosome to two populations of nucleic acids that are distinguishably labeled. In these embodiments, for a single chromosomal structural region oligonucleotide feature, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

By “normalization” is meant that data corresponding to the two populations of nucleic acids are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol. Biol. 4:57-64, 2002, Finkelstein et al, Plant Mol. Biol. 48(1-2): 119-31, 2002, and Hegde et al, Biotechniques. 29:548-554, 2000). Specific examples of normalization suitable for use in the subject methods include linear normalization methods, non-linear normalization methods, e.g., using lowess local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using the data obtained from a support-bound polynucleotide for a chromosome of known ploidy in any of the chromosome compositions.

Accordingly, chromosome copy number may be assessed by detecting binding of one or more chromosomal structural region oligonucleotide features to a labeled population of nucleic acids. In most embodiments, the assessment provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof.

In other words, any binding may be expressed as the level of binding of one or more chromosomal structural region oligonucleotide features to a labeled population of nucleic acids made from a test chromosome composition, divided by its level of binding to a labeled population of nucleic acids made from a reference chromosome composition (or vice versa). This number provides an accurate estimate of chromosome copy number in a cell.

A feature of the above methods is that they are sufficiently sensitive to detect a single copy number difference or change in the amount of a chromosome between any two given cells. In other words, the subject methods are capable of detecting a single copy number variation in a chromosome between any two cells. As such, the subject methods are highly sensitive methods of comparing the copy numbers of one or more chromosomes between two or more cells.

Computer-Related Embodiments

The invention also provides a variety of computer-related embodiments. Specifically, the data analysis methods described in the previous section may be performed using a computer. Accordingly, the invention provides a computer-based system for assessing chromosome copy number using the above methods.

In most embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Kits

Also provided by the subject invention are kits for practicing the subject methods, as described above. The subject kits at least include a chromosomal structural region oligonucleotide that may be surface-bound to a planar solid support. Other optional components of the kit include: nucleic acid labeling agents, such as primer extension or nick translation and fluorescent labels conjugated to nucleotides, Cot-1 or other suppressors or repetitive DNA, and control or reference chromosome compositions for use in testing the other compositions of the kit. In some embodiments, arrays may be included in the kits. In alternative embodiments, the kit may also contain computer-readable media for performing the subject methods, as discussed above. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control analytes for use in testing the kit.

Utility

The above-described compositions and methods find use in any application in which one wishes to compare the copy number of one or more chromosomes in a cell. One type of representative application in which the subject methods find use is the quantitative comparison of copy number of chromosome in a first cell relative to the copy number of the same chromosome in a second cell, i.e., detecting the “ploidy” of a chromosome in a cell.

The subject invention therefore finds use in methods for detecting normal and abnormal chromosome copy number in a cell (e.g., diploidy and euploidy or aneuploidy), and, accordingly, finds particular use as a diagnostic and research tool for investigating diseases and conditions related to chromosomal copy number alterations e.g., genetic abnormalities and cancer.

In general, two populations of labeled nucleic acids, representing a test and reference cells, are hybridized with a subject array as discussed above. The arrays are washed and read to provide data, and that data provides information on the relative copy number of a chromosome in the test and reference cells. In most embodiments, assuming that the reference cell is “normal”, any results that indicate that a particular chromosome is present at a greater amount in a test cell, relative to that of the reference cell, indicates that the chromosome is present at an abnormal copy number in the test cell.

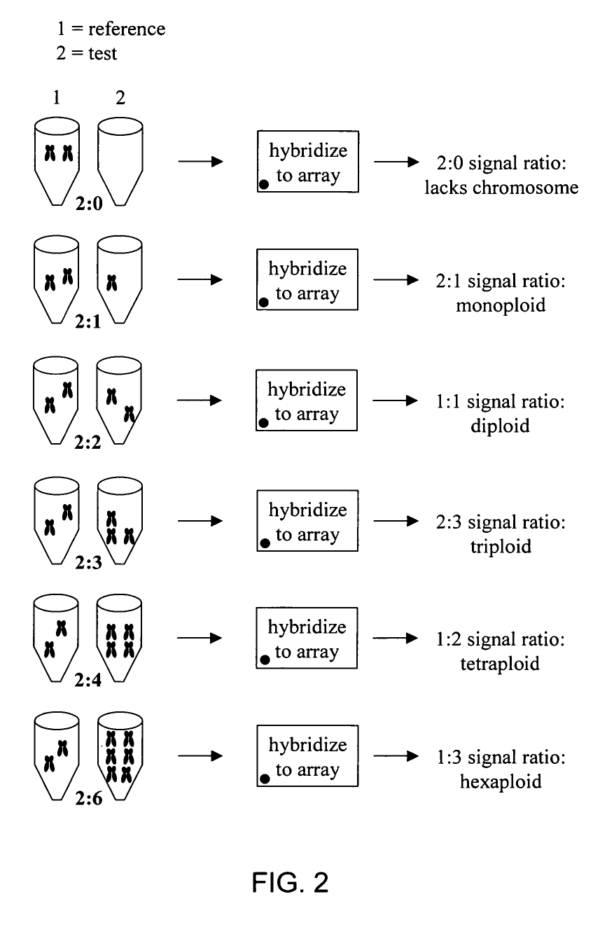

A representative specific utility of the subject methods is illustrated in FIG. 2. This figure shows a series of reference chromosomal compositions (1) from cells that are normally diploid (i.e., having two sets of each chromosome), and a series of test chromosomal compositions from cells of unknown ploidy. Populations of labeled nucleic acids made using the chromosome compositions and hybridized with the subject arrays, and the arrays are ready according to the subject methods. After the data has been normalized, the signal ratios (i.e., the numerical expression of the amount of binding of a subject surface-bound oligonucleotide to each of the populations of labeled nucleic acids) indicate the relative amount of a chromosome in the reference and test cells. In the example set forth in FIG. 2, assays provide signal ratios of 2:0, 2:1, 2:3, 1:2 and 2:6, indicated several test cells as having an abnormal chromosome copy number (i.e. lacking chromosomes, or monosomy, trisomy, tetrasomy, and hexasomy, respectively). One assay provides a signal ratio of 1:1, indicating that the test cell has a chromosome number that is the same as the reference cell, and, is therefore normal and diploid.

Further, the cells used in the subject methods may be a heterogeneous mixture of cells containing chromosomes at different copy number. Accordingly, when such cells are used, a chromosomal copy number may be any number, including any integer and fraction thereof.

The following examples are offered by way of illustration and not by way of limitation.

### EXPERIMENTAL

Materials and Methods

The colon carcinoma line HT 29 was obtained from American Type Culture Collection and grown under conditions specified by the supplier. Genomic DNA was prepared from the cell line using the DNeasy Tissue Kit (Qiagen, Germantown, Md.). Sample labeling. For each CGH hybridization, 20 μg of genomic DNA from the reference (46,XX female) and the corresponding experimental sample was digested with AluI (12.5 units) and RsaI (12.5 units) (Promega). All digests were done for a minimum of 2 hours at 37° C. then verified by agarose gel analysis. Individual reference and experimental samples were then filtered using the Qiaquick PCR Cleanup Kit (Qiagen). Labeling reactions were performed with 6 μg of purified restricted DNA and a Bioprime labeling kit (Invitrogen) according to the manufacturer's directions in a 50 μl volume with a modified dNTP pool; 120 μM each of DATP, dGTP, dTTP, 60 μM dTTP, and 60 μM of either Cy5-dUTP for the experimental sample or Cy3-dUTP for the 46,XX female reference (Perkin-Elmer, Boston, Mass.). Labeled targets were subsequently filtered using a Centricon YM-30 filter (Millipore, Bedford, Mass.). Experimental and reference targets for each hybridization were pooled, mixed with competitor DNA (Invitrogen), 100 μg of yeast tRNA (Invitrogen) and 1× hybridization control targets (SP310, Operon). The target mixture was purified then concentrated with a Centricon YM-30 column, and resuspended to a final volume of 250 μl, then mixed with an equal volume of Agilent 2× in situ Hybridization Buffer.

Results

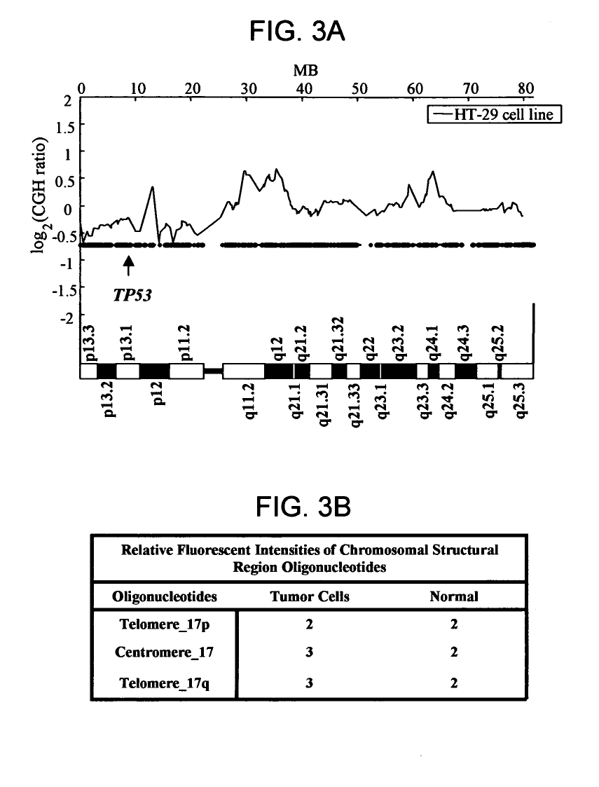

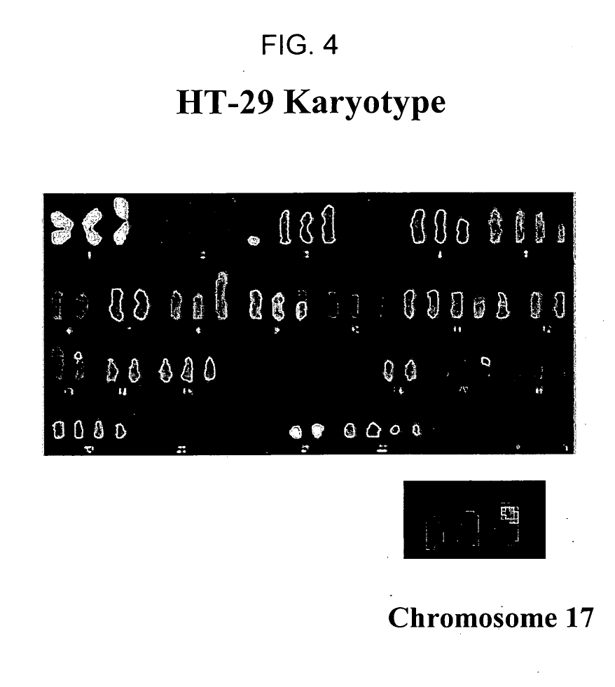

Tumor suppressor genes are typically inactivated by mutation of one copy and loss of the remaining normal copy in tumor cells (Nowell, P C., Science 194, 23-28.(1976). HT-29 colon carcinoma cells contain a known mutation of the TP53 tumor suppressor gene located on the short arm (p) of chromosome 17. The ideal CGH value for loss of a single genomic copy in a diploid cell is a ratio of 0.5 (log₂ ratio=−1.0). CGH analyses detected a log₂ ratio value closer to −0.5 in this region (FIG. 3). However oligonucleotides on the array that detect the copy numbers of the chromosome structural regions detect the presence of 3 copies of the centromere and q arm telomere, and 2 copies of the p arm telomere in these cells. Thus these cells are triploid for chromosome 17 and the ideal CGH ratio for 17p in these cells should approach a log₂ value of −0.6. These data are in agreement with previous cytogenetic analysis that showed HT-29 cells contain 3 copies of chromosome 17, one of which has a truncated p arm (FIG. 4) (Abdel_Rahman et al., (Feb. 20, 2001) Proc Natl Acad Sci USA, 10. 1073). Therefore the simultaneous detection of ploidy status of chromosome 17 with array CGH profiling enables a more accurate description of these regions and the chromosome abnormalities in a single array experiment.

The above results and discussion demonstrate a new method for assessing chromosome copy number in a cell. Such methods are superior to currently used methods because they provide a way of directly and accurately quantifying the number of chromosomes in a cell by counting the number of centromeric and telomeric sequences that are present in the cell, relative to reference cell. Further, because the subject methods rely on oligonucleotides, cross-hybridization can be minimized while maintaining maximum hybridization affinity, and several surface-bound centromeric and/or telomeric oligonucleotides may be straightforwardly designed and used to assay the presence of each chromosome. As such, the subject methods represent a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An array comprising at least one chromosome structural region oligonucleotide feature.

2. The array according to claim 1, wherein said chromosome structural region is chosen from a centromeric region and a telomeric region.

3. The array of claim 1, wherein said array comprises both centromeric and telomeric chromosome structural region oligonucleotide features.

4. The array of claim 1, wherein said array comprises chromosome structural region oligonucleotide features for a plurality of chromosomes of a cell.

5. The array of claim 4, wherein said array comprises a set of chromosome structural region oligonucleotide features for all chromosomes of said cell.

6. The array of claim 1, wherein said array comprises chromosome structural region oligonucleotide features for detecting both telomeres of a chromosome.

7. The array of claim 1, further comprising at least one chromosome non-structural region oligonucleotide feature.

8. The array of claim 7, wherein said at least one chromosome non-structural region oligonucleotide feature is interspersed with said at least one chromosome structural region oligonucleotide feature.

9. The array of claim 8, wherein said at least one chromosome non-structural region oligonucleotide feature is on a separate part of said array to said at least one chromosome structural region oligonucleotide feature.

10. A method for determining chromosome copy number in a cell, comprising:
contacting a first population of labeled nucleic acids made from a chromosomal composition of a test cell with an array of claim 1; and evaluating binding of said nucleic acids to said feature of said array relative to a reference level of binding.

11. The method of claim 10, wherein said reference level of binding is binding of a second population of nucleic acids made from a reference chromosome composition of a reference cell to said feature.

12. The method of claim 11, wherein said test and reference cells are cancerous and non-cancerous cells, respectively.

13. The method of claim 12, wherein said test and reference cells are mammalian cells.

14. The method of claim 13, wherein said test and reference mammalian cells are human cells.

15. The method of claim 10, wherein said array contains chromosome structural region oligonucleotide features for detection of an entire complement of chromosomes of said test cell.

16. The method of claim 10, wherein said contacting step includes contacting said population of nucleic acids to said array under stringent binding conditions.

17. A method for detecting abnormal chromosome copy number in a cell, comprising:
contacting a first population of labeled nucleic acids made from a chromosomal composition of a test cell with an array of claim 1; and
evaluating binding of said nucleic acids to said feature of said array relative to a reference level of binding;
wherein greater or lesser binding of said first population of labeled nucleic acids to said feature as compared to said reference level of binding indicates abnormal chromosome copy number in said test cell.

18. The method of claim 17, wherein said reference level of binding is a level of binding of a second population of nucleic acids made from a reference chromosome composition of a reference cell to said feature.

19. The method of claim 18, wherein said first and second cells are cancerous and non-cancerous cells, respectively.

20. The method of claim 19, wherein said cells are human cells.

21. A method of assaying a sample comprising:
(a) contacting an array according to claim 1 with said sample; and
(b) detecting the presence of binding complexes on the surface of said array to assay said sample.

22. The method according to claim 21, wherein said method is a comparative genomic hybridization assay.

23. A method comprising transmitting data from a method of claim 21 from a first location to a second location.

24. The method of claim 23, wherein said second location is a remote location.

25. A method comprising receiving a transmitted result of a reading of an array obtained according to the method claim 21.

26. A kit for assessing chromosome copy number in a cell, comprising:
an array according to claim 1; and
instructions for using said array to assess chromosome copy number in a cell.

27. The kit of claim 26, further comprising labeling reagents for labeling a chromosomal sample.

28. The kit of claim 26, further comprising a set of primers for labeling a centromere or a telomere of said chromosome.

29. The kit of claim 27, wherein said labeling reagents are for distinguishably labeling two chromosomal samples.

30. A computer-readable medium comprising:
programming for analyzing data provided by the method of claim 10.

31. The computer-readable medium of claim 30, wherein an output of said programming is a chromosome copy number.

32. A computer comprising the computer-readable medium of claim 31.

33. A computer implemented method, comprising:
evaluating data produced by the method of claim 10; and
determining the copy number of a chromosome.
